(19) United States
(12) Patent Application Publication
(10) Pub. No.: US 2017/0052172 A1
(43) Pub. Date: Feb. 23, 2017

(54) IMMUNE HEPATOTOXICITY SCREENING METHOD USING HEPATOCYTES DERIVED FROM HUMAN STEM CELLS

(71) Applicant: KOREA ADVANCED INSTITUTE OF SCIENCE AND TECHNOLOGY, Daejeon (KR)

(72) Inventors: Seung-Hyo LEE, Daejeon (KR); Seyun KIM, Seoul (KR); Yong-Mahn HAN, Daejeon (KR)

(21) Appl. No.: 15/257,338

(22) Filed: Sep. 6, 2016

Related U.S. Application Data
(63) Continuation of application No. PCT/KR2015/002038, filed on Mar. 3, 2015.

(30) Foreign Application Priority Data
Mar. 3, 2014 (KR) 1020140024898

(51) Int. Cl. G01N 33/50; C12N 5/071

(52) U.S. Cl. CPC G01N 33/5067; C12N 5/067; C12N 2506/02; C12N 2501/12

(57) ABSTRACT

The present invention relates to an immune hepatotoxicity screening method using hepatocytes derived from human stem cells. After hepatocytes differentiated from human stem cells and human hepatocytes are treated with ethanol, CCl₄, and acetaminophen to induce immune hepatotoxicity, a hepatocellular immunotoxic material assay system is constructed in order to verify cytokines, chemokines, and lipid mediators, which are mediators secreted from the hepatocytes, and an immunotoxic material can be confirmed in the cells having the induced hepatotoxicity by using the system. Therefore, the immune hepatotoxicity screening method using hepatocytes derived from human stem cells can be favorably used.

### BACKGROUND OF THE INVENTION

1. Field of the Invention

The present invention relates to an immune hepatotoxicity screening method using hepatocytes derived from human stem cells and an immune hepatotoxicity screening kit.

2. Description of the Related Art

The failure ratio in drug development due to toxicity reaches approximately as high as 20% in non-clinical stage and approximately 13% in clinical stage. Even though the toxicity mechanism and the target organ of a new drug candidate that has been thrown out after being registered as a new chemical entity (NCE) are not clearly understood, it is guessed that the heart and the liver together take approximately 42% of the total target organ list in non-clinical stage, according to the reports made by Bristol-Myers Squibb from 1993 to 2006. Drugs which had been thrown out from the market since 1990, because of toxicity, were investigated to find out the reason of being thrown out in clinical stage. As a result, hepatotoxicity was the reason in 13 drugs (about 40%) and heart disease (arrhythmia) due to the increasing QT interval was the reason in 11 drugs (33%), suggesting that the hepatotoxicity and heart toxicity are the two major reasons (74% of the list). At least one of medicines has been kicked out from the market annually because of toxicity since 2004. Among the medicines in clinical use, 515 items in total have been received black box warning (the highest alert to restrict its use) from FDA, USA, most of which have been received warning due to hepatotoxicity and cardiotoxicity.

At least 800 items which are being used as a medicine cause hepatotoxicity, which takes at least 30% of total acute liver failure and takes 2˜20% of hospitalized patients with jaundice in USA. The drug induced liver injury is the major reason of drug development stopping in pre-clinical stage, termination of clinical trial in clinical stage, and being pulled out from a market after commercialized. Various medicines can cause hepatotoxicity, which are exemplified by antibiotics, anti-cancer agents, antihypertensive agents, anticonvulsants, antihyperlipedemic drugs, antipsychotic drugs, nonsteroidal anti-inflammatory drugs, inhalation anesthetics, antidiabetics, and herbal medications. FDA (USA) and European Medicines Agency together continued to make an effort to minimize the drug induced liver injury. The hepatotoxicity inducing mechanism of hepatotoxic drugs depends largely on metabolic activation. FDA (USA) published “guide for industrial safety test of drug metabolites” in February, 2008, emphasizing the importance of the study about drug metabolites in drug development.

Active metabolites were found in 9 out of 14 drugs that had been kicked out because of hepatotoxicity and in 10 out of 14 drugs that had warning labels on them. In general, drug induced hepatotoxicity is usually caused by rather a metabolite derived from a parent drug than the parent drug itself. Among the physiochemical characteristics of a drug metabolite inducing hepatotoxicity, electrophilicity is responsible for mediating hepatotoxicity. Therefore, it is proposed in the early stage of drug development to screen a toxic metabolite having electrophilicity. Chemical structures wherein metabolic activation can cause a problem are varied and exemplified by 1) hydrazine and hydrazide, 2) arylacetic acid and arylpropionic acid, 3) thiophene, furan, and pyrrole, 4) aniline and anilide, 5) quinone and quinoneimine, 6) medium chain fatty acid, 7) halogenated hydrocarbon and halogenated aromatic (Br>Cl>F), 8) nitroaromatic, 9) α,β-unsaturated enol-like structure, 10) thiol or thiono (thiazolidinedione Patent No. 10-1334159 and thiourea), and 11) aminothiazole, etc.

There are diverse problems in hepatotoxicity evaluation system in drug development. First, a specific target for the prediction of hepatotoxicity is not identified, yet. For example, in the case of cardiotoxicity, HERG channel assay was proposed as a standard for the evaluation of cardiotoxicity, and in vivo effect and involvement of HERG channel have been reported. However, it is very difficult to establish an evaluation system with a specific protein as a target for hepatotoxicity.

Second, it is still difficult to predict human hepatotoxicity with the animal test results. For example, 31 new drug candidates out of 238 candidates were confirmed to cause hepatotoxicity in the study of international life sciences institute in 1999, and only 58% of it showed hepatotoxicity in animal test, though, indicating that animal test result provided low predictability. In addition, there was a big difference in hepatotoxicity evaluation by Rhone-Roulenc Rorer between the animal test and the clinical test.

Third, idiosyncratic hepatotoxicity was observed in hypersensitive individuals. The drugs withdrawn from a market because of hepatotoxicity are characteristically not clear in their dose dependence and mechanism and particularly cause more severe toxicity in hypersensitive individuals (one of 10,000 or 100,000 people). Therefore, it might not be identified until phase 3 clinical trial or not even in NDA (new drug application) process.

Fourth, the prediction of hepatotoxicity with the traditional animal test does not meet the requirement of drug development industry. The quantitative and qualitative differences in drug metabolite pattern make a big difference in interspecific hepatotoxicity. Besides, the traditional hepatotoxicity evaluation system could not predict the hepatotoxicity of those drugs that were thrown out of the market due to idiosyncratic liver injury.

Fifth, human liver cancer cell line HepG2, transformed HepG2 (drug metabolizing enzyme (cytochrome P450, CYP) over-expressing cell line), immortalized human hepatocytes (insertion of SV40 T antigen gene), and primary hepatocytes (human or rat) are used in a cell culture system as a hepatotoxicity prediction model. However, all of them are limited in predicting hepatotoxicity in clinical stage.

Thus, the present inventors tried to develop an immune hepatotoxicity screening method using hepatocytes derived from human stem cells. After hepatocytes differentiated from human stem cells and human hepatocytes were treated with ethanol, CCl₄, and acetaminophen to induce immune hepatotoxicity, a hepatocellular immunotoxic material assay system was constructed in order to verify cytokines, chemokines, and lipid mediators, which are mediators secreted from the hepatocytes, and an immunotoxic material could be confirmed in the cells having the induced hepatotoxicity by using the system above. It was additionally confirmed that the human hepatocyte like cells of the invention were useful for the evaluation of metabolism or hepatotoxicity of a drug candidate compound without an animal model. Therefore, the hepatocytes of the invention derived from human stem cells were confirmed to be useful for the immune hepatotoxicity screening method and for the hepatotoxicity inducing material screening kit, leading to the completion of the invention.

### SUMMARY OF THE INVENTION

It is an object of the present invention to provide an immune hepatotoxicity screening method using the hepatocytes derived from human stem cells

It is another object of the present invention to provide an immune hepatotoxicity screening kit using the hepatocytes derived from human stem cells.

To achieve the above objects, the present invention provides a hepatotoxicity inducing material screening method comprising the following steps:

1) treating the test material to the hepatocytes differentiated from human stem cells; and

2) measuring the level of apoptosis or proliferation inhibition in the hepatocytes of step 1).

The present invention also provides a hepatotoxicity inducing material screening method comprising the following steps:

1) treating the test material to the hepatocytes differentiated from human stem cells;

2) obtaining the culture fluid of the hepatocytes of step 1) or the supernatant of the same; and

3) analyzing cytokines, chemokines, or lipid mediators in the culture fluid or the supernatant of step 2).

The present invention further provides a hepatotoxicity inducing material detection kit comprising the hepatocytes differentiated from human stem cells.

The present invention also provides a hepatotoxicity inducing material detection kit comprising the hepatocytes differentiated from human stem cells and the detection reagent to detect the mediators secreted from the hepatocytes.

The present invention also provides a use of the hepatotoxicity inducing material detection kit comprising the hepatocytes differentiated from human stem cells.

In addition, the present invention provides a use of the hepatotoxicity inducing material detection kit comprising the hepatocytes differentiated from human stem cells and the detection reagent to detect the mediators secreted from the hepatocytes.

### Advantageous Effect

The present invention relates to an immune hepatotoxicity screening method using hepatocytes derived from human stem cells. After hepatocytes differentiated from human stem cells and human hepatocytes are treated with ethanol, CCl₄, and acetaminophen to induce immune hepatotoxicity, a hepatocellular immunotoxic material assay system is constructed in order to verify cytokines, chemokines, and lipid mediators, which are mediators secreted from the hepatocytes, and an immunotoxic material can be confirmed in the cells having the induced hepatotoxicity by using the system. Therefore, the immune hepatotoxicity screening method using hepatocytes derived from human stem cells can be favorably used.

### BRIEF DESCRIPTION OF THE DRAWINGS

The application of the preferred embodiments of the present invention is best understood with reference to the accompanying drawings, wherein:

FIG. 1 is a schematic diagram illustrating the differentiation of hepatocytes from human pluripotent embryonic stem cells.

FIG. 2 is a schematic diagram illustrating the first and the second inducement of hepatotoxicity.

FIG. 3 is a diagram illustrating the construction of an immunotoxic material ELISA assay system.

FIG. 4 is a diagram illustrating the results of the immunotoxic material ELISA assay with the hepatocytes differentiated from human stem cells and human hepatocytes according to the treatment of ethanol.

FIG. 5 is a diagram illustrating the results of the immunotoxic material ELISA assay with the hepatocytes differentiated from human stem cells and human hepatocytes according to the treatment of CCL₄.

FIG. 6 is a diagram illustrating the results of the immunotoxic material ELISA assay with the hepatocytes differentiated from human stem cells and human hepatocytes according to the treatment of acetaminophen.

### DESCRIPTION OF THE PREFERRED EMBODIMENTS

Hereinafter, the present invention is described in detail.

The present invention provides a hepatotoxicity inducing material screening method comprising the following steps:

1) treating the test material to the hepatocytes differentiated from human stem cells; and

2) measuring the level of apoptosis or proliferation inhibition in the hepatocytes of step 1).

In the method above, the human stem cells in step 1) are preferably human embryonic stem cells, but not always limited thereto.

In the method above, the differentiation of step 1) is induced by the following steps, but not always limited thereto:

a) inducing the differentiation of human stem cells by culture; and

b) culturing the differentiated cells above in a medium supplemented with a hepatocyte growth factor.

In the method above, the hepatotoxicity inducing material is one of the materials selected from the group consisting of ethanol, CCl₄, and acetaminophen, but not always limited thereto.

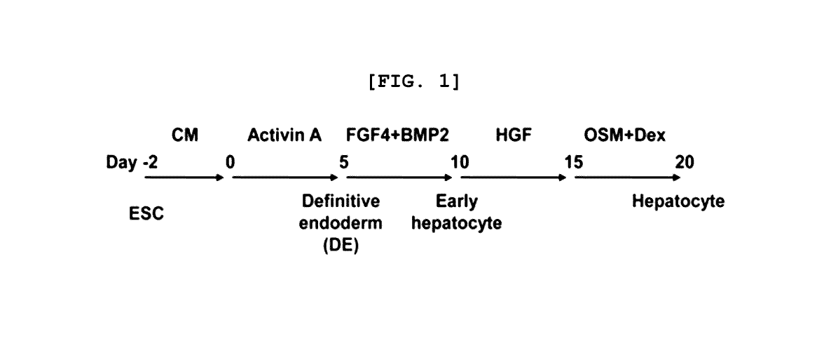

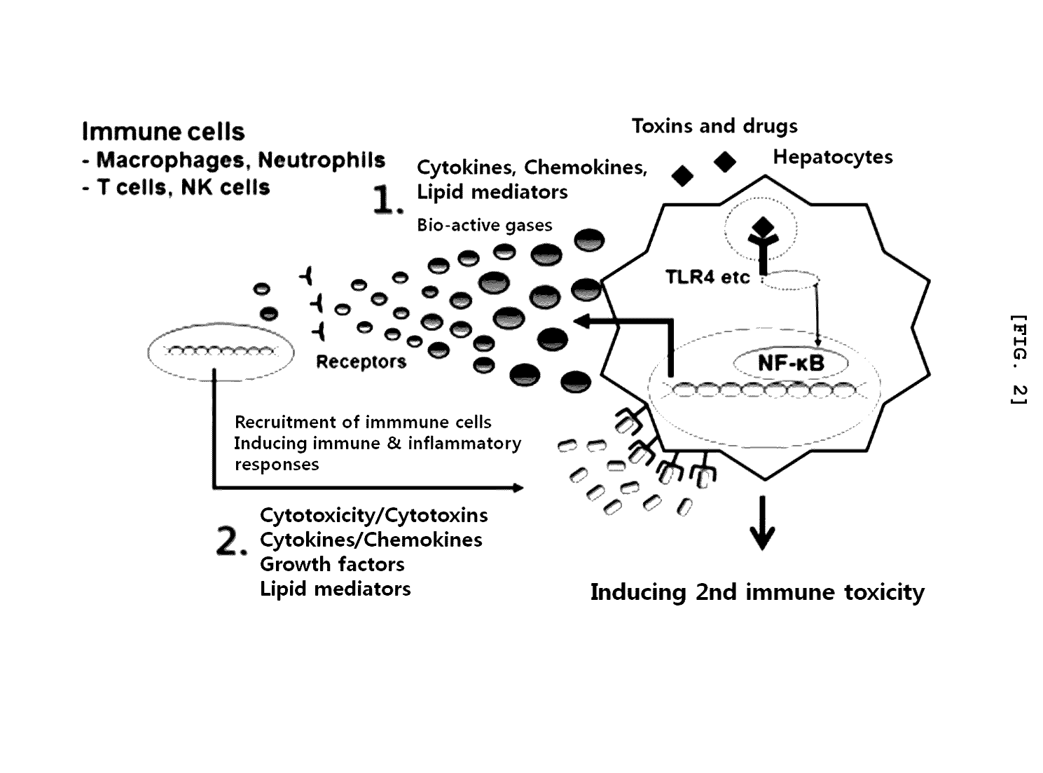

In a preferred embodiment of the present invention, the differentiation of human embryonic stem cells (CHA-hESC4) into hepatocytes (endoderm) was induced in order to investigate the possibility of the differentiation of human embryonic stem cells into other differentiation lineage cells such as endoderm. To compare the immunotoxicity caused in the cells above by a toxic material, human pluripotent embryonic stem cells (CHA-hESC15) was differentiated into hepatocytes by the method shown in the schematic diagram of FIG. 1. The differentiated hepatocytes were cultured in the hepatocyte culture medium for 5 days for the growth of the hepatocytes. The mature hepatocytes were obtained.

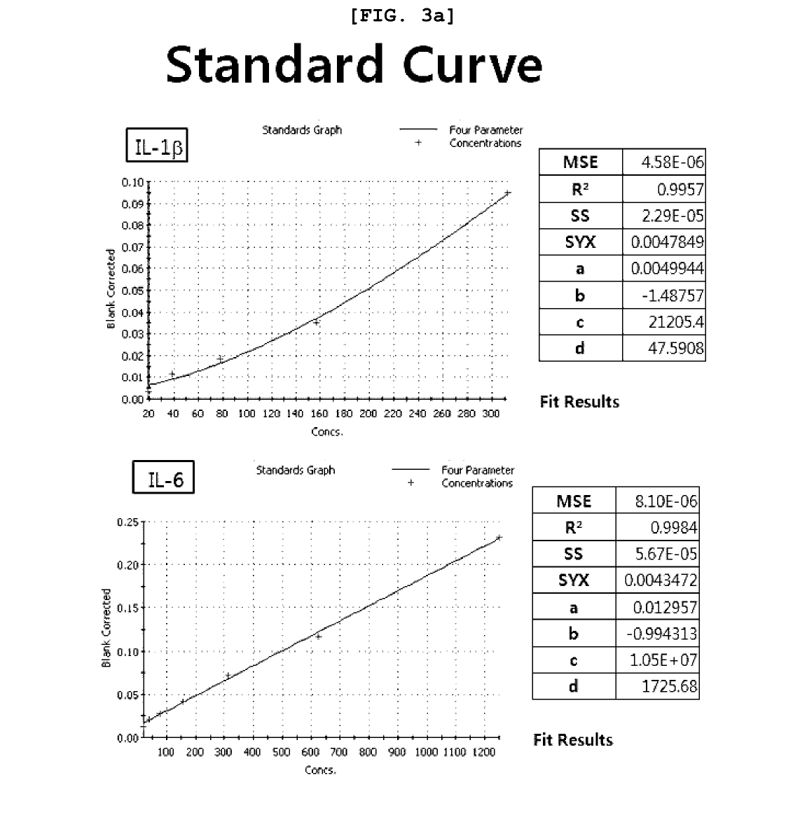

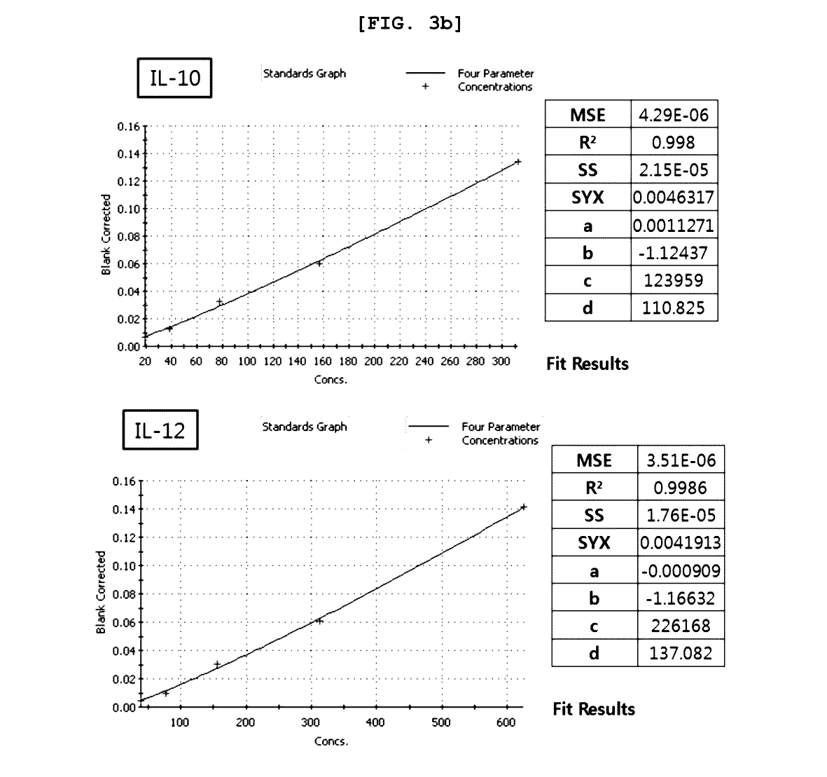

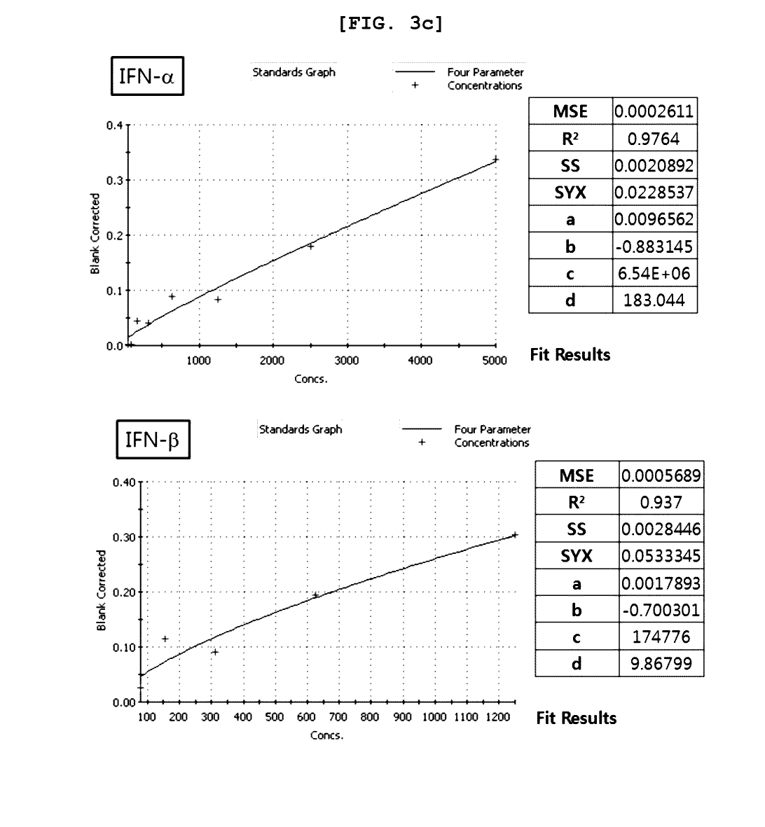

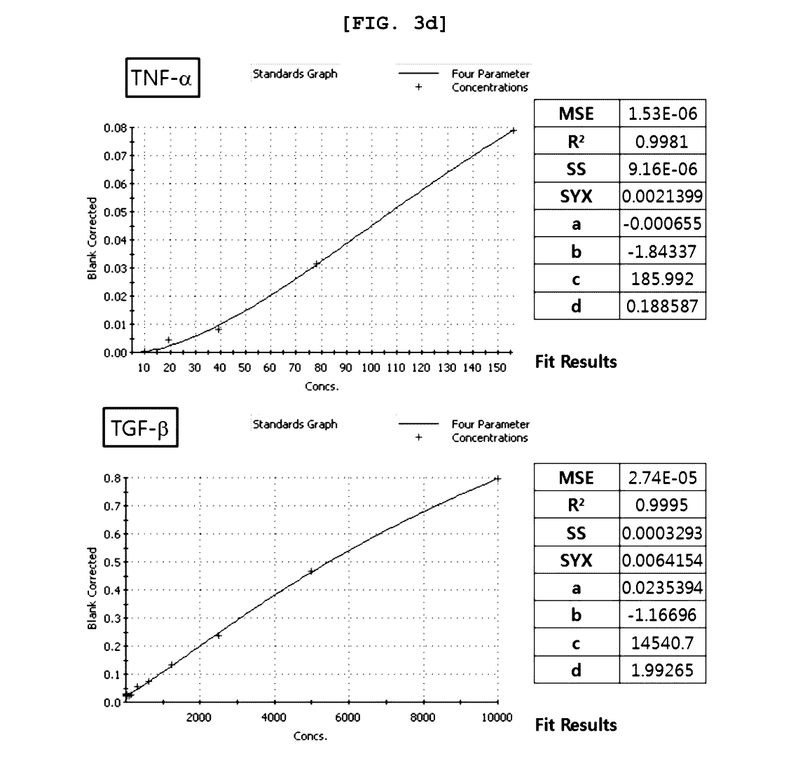

Hepatotoxic reaction is generally divided into two types; the primary toxic reaction that is caused directly by a drug and a toxic material; and the secondary immunotoxic reaction that is induced by the metabolites generated from the reaction with a drug through immune cell activation and inflammatory reaction. To investigate immunotoxicity, it is necessary to analyze such metabolites as protein, lipid, and bioactive gas, etc, generated by hepatocytes (see FIG. 2). Thus, ELISA assay was prepared for 8 kinds of pro-inflammatory cytokine immunotoxic materials such as human IL-1β, IL-6, IL-10, IL-12, TNF-α, IFN-α/β, and TGF-β. ELISA was performed with different concentrations of each cytokine and each antibody against those cytokines and the optimum condition was established (see FIG. 3).

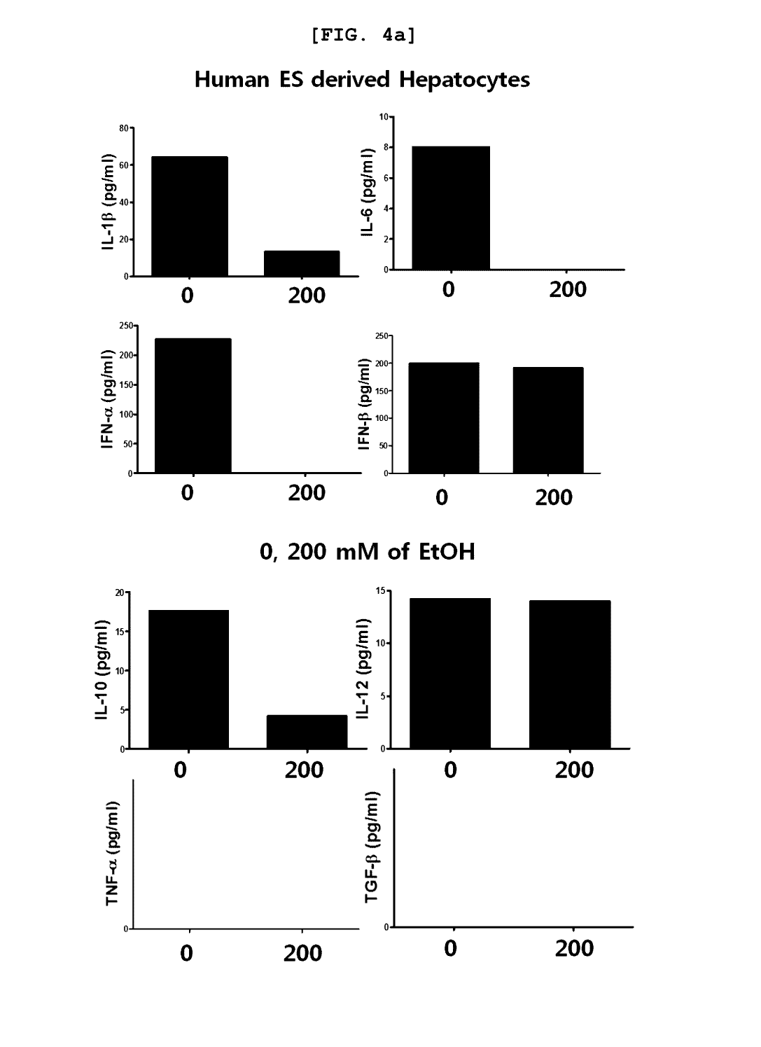

To investigate the drug induced immunotoxicity in the hepatocytes differentiated from human stem cells and human hepatocytes, the cells were treated with ethanol at different concentrations. As a result, pro-inflammatory cytokine was not observed in the hepatocytes differentiated from human stem cells but was detected some in human hepatocytes after the treatment of ethanol. Therefore, it was suggested that the hepatocytes differentiated from human stem cells did not show toxic reaction against ethanol, like the result of general toxicity test (see FIG. 4).

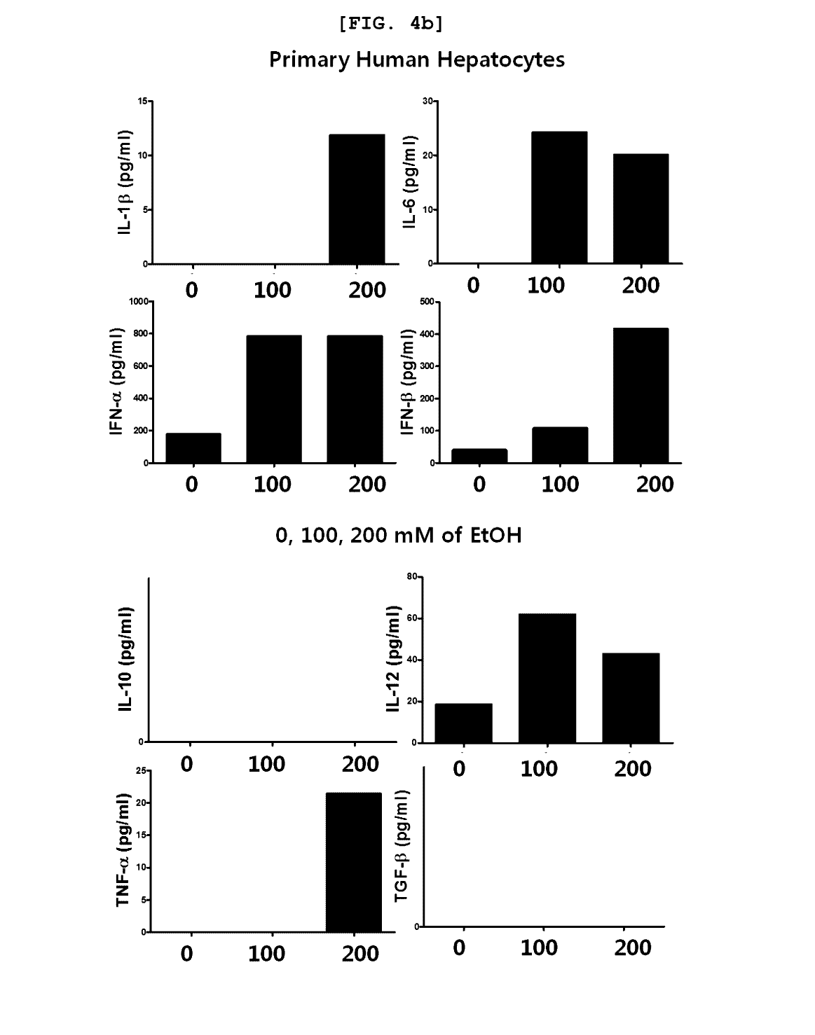

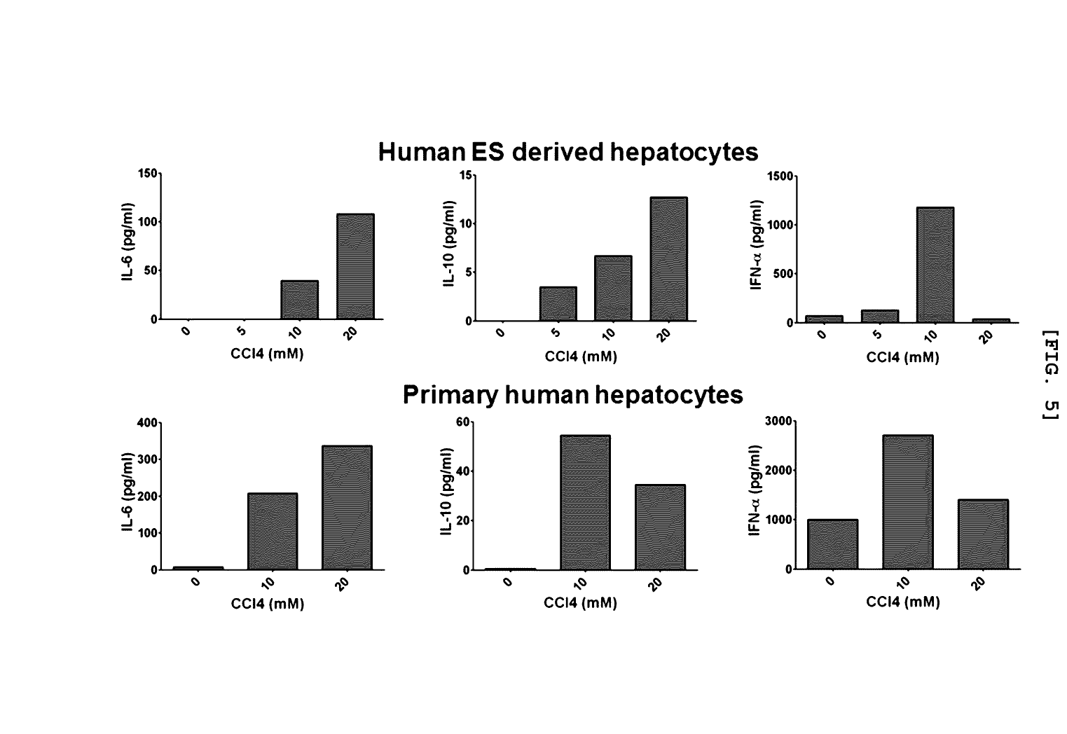

To analyze drug induced immunotoxicity in the hepatocytes differentiated from human embryonic stem cells and human hepatocytes, the hepatocytes differentiated from human stem cells (hESC4) were treated with CCl₄. As a result, IL-6, IL-10, and IFN-α were detected in the cells CCl₄ dose dependently. Human hepatocytes (hESC15) were also treated with CCl₄. As a result, IL-6, IL-10, and IFN-α were detected in the cells as well after the culture, which were though higher levels than those in the hepatocytes differentiated from stem cells, suggesting that cytokines were secreted more in human hepatocytes (see FIG. 5).

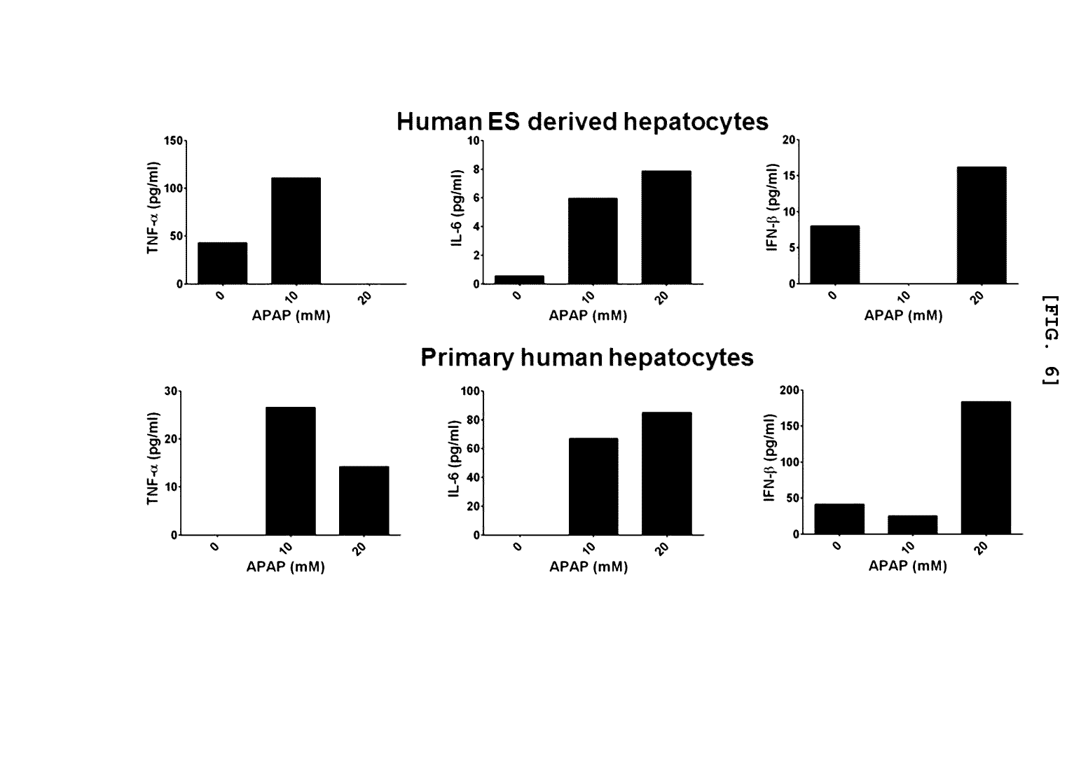

To analyze drug induced immunotoxicity in the hepatocytes differentiated from human stem cells and human hepatocytes, the hepatocytes differentiated from human embryonic stem cells (hESC4) were treated with acetaminophen. As a result, IL-6, TNF-α, and IFN-β were detected in the cells acetaminophen dose dependently. Also, human hepatocytes were treated with acetaminophen. As a result, IL-6, TNF-α, and IFN-β were detected in the cells. Considering that the number of the human hepatocytes was smaller than the number of the hepatocytes differentiated from human stem cells, cytokines such as IL-6 and IFN-β were secreted more in human hepatocytes. On the other hand, TNF-α was highly secreted in the hepatocytes differentiated from stem cells (see FIG. 6).

After the hepatocytes differentiated from human stem cells and human hepatocytes were treated with ethanol, CCl₄, and acetaminophen to induce hepatotoxicity, a hepatocellular immunotoxic material assay system was constructed in order to verify cytokines, chemokines, and lipid mediators, which are mediators secreted from the hepatocytes. An immunotoxic material could be confirmed in the cells having the induced hepatotoxicity by using the system, so that the system can be effectively used as an immune hepatotoxicity screening method using the hepatocytes derived from human stem cells.

The present invention also provides a hepatotoxicity inducing material screening method comprising the following steps:

1) treating the test material to the hepatocytes differentiated from human stem cells;

2) obtaining the culture fluid of the hepatocytes of step 1) or the supernatant of the same; and

3) analyzing cytokines, chemokines, or lipid mediators in the culture fluid or the supernatant of step 2).

In the method above, the stem cells of step 1) are preferably human embryonic stem cells, but not always limited thereto.

In the method above, the cytokine of step 3) is preferably selected from the group consisting of IL-1β, IL-6, IL-10, IL-12, TNF-α, IFN-α, and IFN-β, but not always limited thereto.

In the method above, the chemokine of step 3) is preferably selected from the group consisting of IL-8, IP-10, RANTES, MIP-1, and MCP, but not always limited thereto.

In the method above, the lipid mediator of step 3) is preferably selected from the group consisting of prostaglandin and leukotriene, but not always limited thereto.

In the method above, the hepatotoxicity inducing material is preferably selected from the group consisting of ethanol, CCl₄, and acetaminophen, but not always limited thereto.

After the hepatocytes differentiated from human stem cells and human hepatocytes were treated with ethanol, CCl₄, and acetaminophen to induce hepatotoxicity, a hepatocellular immunotoxic material assay system was constructed in order to verify cytokines, chemokines, and lipid mediators, which are mediators secreted from the hepatocytes. An immunotoxic material could be confirmed in the cells having the induced hepatotoxicity by using the system, so that the system can be effectively used as a hepatotoxicity inducing material screening method.

The present invention further provides a hepatotoxicity inducing material detection kit comprising the hepatocytes differentiated from human stem cells.

The present invention also provides a hepatotoxicity inducing material detection kit comprising the hepatocytes differentiated from human stem cells and the detection reagent to detect the mediators secreted from the hepatocytes.

The present invention also provides a use of the hepatotoxicity inducing material detection kit comprising the hepatocytes differentiated from human stem cells.

In addition, the present invention provides a use of the hepatotoxicity inducing material detection kit comprising the hepatocytes differentiated from human stem cells and the detection reagent to detect the mediators secreted from the hepatocytes.

In the method above, the hepatotoxicity inducing material is preferably selected from the group consisting of ethanol, CCl₄, and acetaminophen, but not always limited thereto.

In the method above, the mediator is preferably selected from the group consisting of cytokines, chemokines, and lipid mediators, but not always limited thereto.

In the method above, the mediator detection reagent is preferably selected from the group consisting of those detection reagents listed in Table 1 below, but not always limited thereto.

TABLE 1

| | | | | Detection |
|---|---|---|---|---|
| | cytokine | Standard protein | Capture antibody | antibody |
| BioLegend | IL-1β | Catalog#: 579409 | Clone: H1b-27 | Clone: H1b-98 |
| | | | Catalog#: 511602 | Catalog#: 511704 |
| BioLegend | IL-10 | Catalog#: 571009 | Clone: JES3-907 | Clone: JES3-12G8 |
| | | | Catalog#: 501402 | Catalog#: 501502 |
| BioLegend | IL-12/23 (p40) | Catalog#: 572109 | Clone: C8.3 | Clone: C8.6 |
| | | | Catalog#: 501702 | Catalog#: 508802 |
| BioLegend | TNF-α | Catalog#: 570109 | Clone: MAb1 | Clone: MAb11 |
| | | | Catalog#: 502802 | Catalog#: 502904 |
| Peprotech | IFN-α | Catalog#: 300-02A | Catalog#: | Catalog#: |
| | | | 500-P32AG | 500-P32Abt |
| Peprotech | IFN-β | Catalog#: 300-02BC | Catalog#: | Catalog#: |
| | | | 500-P32B | 500-P32Bbt |
| B&D | TGF-β1 | Catalog#: 240-B-010 | Clone: #9016 | Purified |
| | | | Catalog#: MAB240 | polyclonal |
| | | | | chicken IgY |
| | | | | Catalog#: BAF240 |
| BD | IL-6 | Catalog#: 560071 | Clone: MQ2-13A5 | Clone: MQ2-39C3 |
| Biosciences | | | Catalog#: 554543 | Catalog#: 564546 |

After the hepatocytes differentiated from human stem cells and human hepatocytes were treated with ethanol, CCl₄, and acetaminophen to induce hepatotoxicity, a hepatocellular immunotoxic material assay system was constructed in order to verify cytokines, chemokines, and lipid mediators, which are mediators secreted from the hepatocytes. An immunotoxic material could be confirmed in the cells having the induced hepatotoxicity by using the system, so that the system can be effectively used as a hepatotoxicity inducing material screening kit.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Differentiation of Hepatocytes from Human Embryonic Stem Cells

To investigate the possibility of the differentiation of human embryonic stem cells into other differentiation lineage cells such as endoderm, the differentiation of human embryonic stem cells into hepatocytes, the endodermal cells, was induced (Cai, J. et. al, (2007) Hepatology 45(5): 1229-1239.).

Particularly, as a hESC cell line, CHA-hESC4 cell line was cultured in a conditioned medium in feeder-free system for 3 days until it would be confluent. Upon completion of the culture, the hESC cells were cultured in RPMI-1640 (Hyclone, USA) containing 50 ng/ml of Activin A (Peprotech, USA) for 5 days to induce differentiation. Then, the differentiated cells were cultured in the hepatocyte culture medium (HCM; Lonza, USA) supplemented with 30 ng/ml of fibroblast growth factor 4 (Peprotech) and 20 ng/ml of bone morphogenetic protein 2 (BMP2; Peprotech) for another 5 days. The cells were further cultured in the hepatocyte culture medium supplemented with 20 ng/ml of hepatocyte growth factor (HGF; Peprotech) to induce differentiation of hESC into hepatocytes. The differentiated hepatocytes were cultured in the hepatocyte culture medium supplemented with 10 ng/ml of oncostatin M (R&D Systems, USA) and 0.1 μM dexametasone (Sigma-Aldrich, USA) to induce maturation. Then, the matured hepatocytes were obtained.

### Example 2

### Differentiation of Hepatocytes from Human Pluripotent Embryonic Stem Cells

The following experiment was performed to induce the differentiation of human pluripotent embryonic stem cells into hepatocytes.

Particularly, as shown in FIG. 1, as a hESC cell line, CHA-hESC15 cell line was cultured in a conditioned medium in feeder-free system for 3 days until it would be confluent. Upon completion of the culture, the maturation of hepatocytes was induced by the same manner as described in Example 1 and then the matured hepatocytes were obtained (FIG. 1).

### Example 3

### Construction of Hepatocellular Immunotoxic Material Assay System

Hepatotoxic reaction is generally divided into two types; the primary toxic reaction that is caused directly by a drug and a toxic material; and the secondary immunotoxic reaction that is induced by the metabolites generated from the reaction with a drug through immune cell activation and inflammatory reaction. To investigate immunotoxicity, it is necessary to analyze such metabolites as protein, lipid, and bioactive gas, etc, generated by hepatocytes (FIG. 2). So, the following experiment was performed to construct an immunotoxic material assay system to analyze pro-inflammatory cytokines.

Particularly, the hepatocytes at 19th day of the differentiation from human stem cells, which were prepared by the method described in Example 1, were distributed in a 6-well plate (90%/well), and then the medium was replaced with 1000 mg/L DMEM-low glucose medium (Dulbecco's Modified Eagle's Medium, Welgene No. 001-11), followed by culture for 2 hours. Then, the stem cell derived hepatocyte culture medium was obtained. To eliminate by-products of the cell culture, centrifugation was performed at 12,000×g, for 20 minutes at 4° C. Supernatant was obtained. The capture antibodies, 2.5 μg/ml anti-IL-1β antibody, 2 μg/ml anti-IL-6 antibody, 2.5 μg/ml anti-IL-10 antibody, 2 μg/ml anti-IL-12 antibody, 1 μg/ml anti-TNF-α antibody, 2 μg/ml anti-IFN-α antibody, 1 μg/ml anti-IFN-β antibody, and 2 μg/ml anti-TGF-β antibody, were cultured at 37° C. for 2 hours, with which 96-well ELISA plate was coated. The plate was washed, followed by blocking with 0.2% I-block (Foster city, CA) at 37° C. for 2 hours. Upon completion of the blocking, the capture antibodies were washed and cultured with a standard material (2 fold serial diluted in protein free flexible plate from 10 ng/ml to 4.88 pg/ml) and the supernatant in ELISA plate (Greiner, Kremsmunster, Austria) at 37° C. for 2 hours. Upon completion of the culture, the biotinylated detection antibodies (anti-IL-1β antibody: 2 μg/ml, anti-IL-6 antibody: 1 μg/ml, anti-IL-10 antibody: 1 μg/ml, anti-IL-12 antibody: 1 μg/ml, anti-TNF-α antibody: 1 μg/ml, anti-IFN-α antibody: 4 μg/ml, anti-IFN-β antibody: 2 μg/ml, anti-TGF-β antibody: 1 μg/ml, final concentration) were added thereto, followed by further culture at 37° C. for 2 hours. Then, alkaline phosphatase conjugated streptavidin was added thereto, followed by further culture at room temperature for 30 minutes. 5 mM nitrophenyl phosphate substrate was added thereto, and OD₄₀₅ was measured by using ELISA reader.

As a result, as shown in FIG. 3, ELISA assay was prepared for 8 kinds of immunotoxic materials such as human IL-1β, IL-6, IL-10, IL-12, TNF-α, IFN-α/β, and TGF-β, which were pro-inflammatory cytokines. Each cytokine and its corresponding antibody were secured, followed by dose-dependent ELISA assay and accordingly the optimum condition was determined. To confirm the ELISA assay construction, the standard curve of each cytokine was analyzed, by which R² value was confirmed to be at least 0.9 (FIG. 3).

### Example 4

### Analysis of an Immunotoxic Material by Treating the Differentiated Hepatocytes with a Drug

### <4-1> Confirmation of an Immunotoxic Material by Ethanol Treatment

The hepatocytes differentiated from human embryonic stem cells by the method described in Example 1 were treated with a drug and then the immunotoxic material was analyzed by the following method.

Particularly, the hepatocytes at 19^{th} day of the differentiation from human stem cells, which were prepared by the method described in Example 1, were distributed in a 6-well plate (90%/well), and then the medium was replaced with 1000 mg/L DMEM-low glucose medium (Dulbecco's Modified Eagle's Medium, Welgene No. 001-11), followed by culture for 2 hours. The cultured cells were treated with ethanol (Merk, No. 100983) at different concentrations of 0, 100, and 200 mM for 24 hours. To compare the production of pro-inflammatory cytokines induced by the treatment of ethanol in the hepatocytes differentiated from stem cells and human hepatocytes, the human hepatocytes cultured by the same manner as described in Example 2 were treated with ethanol (Merk, No. 100983) at different concentrations of 0, 100, and 200 mM for 24 hours. Then, culture fluids of the hepatocytes differentiated from stem cells and the human hepatocytes were obtained to extract immunotoxic materials therefrom. To eliminate by-products of the cultured cells, centrifugation was performed at 12,000×g for 20 minutes at 4° C., and then supernatant was obtained. Ethanol dependent hepatocellular cytokine secretion profiling was performed by using the cytokine ELISA assay system constructed by the method described in Example 3.

As a result, as shown in FIG. 4, it was confirmed that pro-inflammatory cytokines were not generated in the hepatocytes differentiated from human stem cells by the treatment of ethanol regardless of the concentration. However, cytokine was detected some in the human hepatocytes cultured by the method described in Example 2 according to the treatment of ethanol. Therefore, consistently with the result of general toxicity test, the hepatocytes differentiated from stem cells did not show ethanol induced toxic reaction (FIG. 4).

### <4-2> Confirmation of an Immunotoxic Material by CCL₄ Treatment

The hepatocytes differentiated from human embryonic stem cells by the method described in Example 1 were treated with a drug and then the immunotoxic material was analyzed by the following method.

Particularly, the hepatocytes differentiated from human embryonic stem cells (hESC4) by the method described in Example 1 were treated with CCl₄ (stock prepared by mixing CCl₄ and DMSO at the ratio of 1:1, Sigma, No. 319961) at different concentrations of 0, 5, 10, and 20 mM for 24 hours. To compare the production of pro-inflammatory cytokines induced by the treatment of CCl₄ in the hepatocytes differentiated from stem cells and human hepatocytes, the human hepatocytes cultured by the same manner as described in Example 2 were treated with CCl₄ (Sigma, No. 319961) at different concentrations of 0, 5, 10, and 20 mM for 24 hours. Then, culture fluids of the hepatocytes differentiated from stem cells and the human hepatocytes were obtained to extract immunotoxic materials therefrom. To eliminate by-products of the cultured cells, centrifugation was performed at 12,000×g for 20 minutes at 4° C., and then supernatant was obtained. CCl₄ dependent hepatocellular cytokine secretion profiling was performed by using the cytokine ELISA assay system constructed by the method described in Example 3.

As a result, as shown in FIG. 5, when CCl₄ was treated to the hepatocytes differentiated from human embryonic stem cells (hES4), IL-6, IL-10, and IFN-α were detected in the cells CCl₄ dose dependently. When human hepatocytes (hES15) were treated with CCl₄, IL-6, IL-10, and IFN-α were also detected in the cells. Considering that the number of the human hepatocytes was smaller than the number of the hepatocytes differentiated from stem cells, cytokines were secreted more in human hepatocytes (FIG. 5).

### <4-3> Confirmation of an Immunotoxic Material by Acetaminophen Treatment

The hepatocytes differentiated from human embryonic stem cells by the method described in Example 1 were treated with a drug and then the immunotoxic material was analyzed by the following method.

Particularly, the hepatocytes differentiated from human embryonic stem cells (hESC4) by the method described in Example 1 were treated with 1.75 M acetaminophen (dissolved in DMSO, Sigma, No. A5000) at different concentrations of 0, 10, and 20 mM for 24 hours. To compare the production of pro-inflammatory cytokines induced by the treatment of acetaminophen in the hepatocytes differentiated from stem cells and human hepatocytes, the human hepatocytes cultured by the same manner as described in Example 2 were treated with acetaminophen at different concentrations of 0, 10, and 20 mM for 24 hours. Then, culture fluids of the hepatocytes differentiated from stem cells and the human hepatocytes were obtained to extract immunotoxic materials therefrom. To eliminate by-products of the cultured cells, centrifugation was performed at 12,000×g for 20 minutes at 4° C., and then supernatant was obtained. Acetaminophen dependent hepatocellular cytokine secretion profiling was performed by using the cytokine ELISA assay system constructed by the method described in Example 3.

As a result, as shown in FIG. 6, when acetaminophen was treated to the hepatocytes differentiated from human embryonic stem cells (hESC4), IL-6, TNF-α, and IFN-β were detected in the cells acetaminophen dose dependently. When human hepatocytes (hES15) were treated with acetaminophen, IL-6, TNF-α, and IFN-β were also detected in the cells. Considering that the number of the human hepatocytes was smaller than the number of the hepatocytes differentiated from human stem cells, cytokines such as IL-6 and IFN-β were secreted more in human hepatocytes. On the other hand, TNF-α was highly secreted in the hepatocytes differentiated from stem cells (FIG. 6).

Those skilled in the art will appreciate that the conceptions and specific embodiments disclosed in the foregoing description may be readily utilized as M a basis for modifying or designing other embodiments for carrying out the same purposes of the present invention. Those skilled in the art will also appreciate that such equivalent embodiments do not depart from the spirit and scope of the invention as set forth in the appended Claims.

1. A hepatotoxicity inducing material screening method comprising the following steps:
1) treating the test material to the hepatocytes differentiated from human stem cells; and
2) measuring the level of apoptosis or proliferation inhibition in the hepatocytes of step 1).

2. The hepatotoxicity inducing material screening method according to claim 1, wherein the human stem cells of step 1) are human embryonic stem cells.

3. The hepatotoxicity inducing material screening method according to claim 1, wherein the differentiation of step 1) is induced by the following steps:
a) inducing the differentiation of human stem cells by culture; and
b) culturing the differentiated cells above in a medium supplemented with a hepatocyte growth factor.

4. A hepatotoxicity inducing material screening method comprising the following steps:
1) treating the test material to the hepatocytes differentiated from human stem cells;
2) obtaining the culture fluid of the hepatocytes of step 1) or the supernatant of the same; and
3) analyzing cytokines, chemokines, or lipid mediators in the culture fluid or the supernatant of step 2).

5. The hepatotoxicity inducing material screening method according to claim 4, wherein the human stem cells of step 1) are human embryonic stem cells.

6. The hepatotoxicity inducing material screening method according to claim 4, wherein the cytokine of step 3) is selected from the group consisting of IL-1β, IL-6, IL-10, IL-12, TNF-α, IFN-α, IFN-β, and TGF-β.

7. The hepatotoxicity inducing material screening method according to claim 4, wherein the chemokine of step 3) is selected from the group consisting of IL-8, IP-10, RANTES, MIP-1, and MCP.

8. The hepatotoxicity inducing material screening method according to claim 4, wherein the lipid mediator of step 3) is selected from the group consisting of prostaglandin and leukotriene.

9. The hepatotoxicity inducing material screening method according to claim 1, wherein the hepatotoxicity inducing material is selected from the group consisting of ethanol, CCl₄, and acetaminophen.

10. A hepatotoxicity inducing material detection kit comprising the hepatocytes differentiated from human stem cells.

11. The hepatotoxicity inducing material detection kit according to claim 10, wherein the hepatotoxicity inducing material is selected from the group consisting of ethanol, CCl₄, and acetaminophen.

12. The hepatotoxicity inducing material detection kit according to claim 10, further comprising a detection reagent to detect the mediators secreted from the hepatocytes.

13.-14. (canceled)

15. The hepatotoxicity inducing material screening method according to claim 4, wherein the hepatotoxicity inducing material is selected from the group consisting of ethanol, CCl₄, and acetaminophen.

16. The hepatotoxicity inducing material detection kit according to claim 12, wherein the hepatotoxicity inducing material is selected from the group consisting of ethanol, CCl₄, and acetaminophen.
